(19) United States
(12) Patent Application Publication
(10) Pub. No.: US 2006/0110781 A1
(43) Pub. Date: May 25, 2006

(54) Cholesterol detection reagent

(75) Inventors: Toshihide Kobayashi, Tokyo (JP); Satoshi Sato, Kyoto (JP); Yoshio Hamashima, Kyoto (JP)

Correspondence Address: GREENBLUM & BERNSTEIN, P.L.C., 1950 ROLAND CLARKE PLACE, RESTON, VA, 20191, US

(73) Assignee: RIKEN, Saitama (JP)

(21) Appl. No.: 10/516,072

(22) PCT Filed: May 30, 2003
(86) PCT No.: PCT/JP03/06841

§ 371 Date: Dec. 21, 2005

(30) Foreign Application Priority Data
May 31, 2002 (JP) 2002-160277

(51) Int. Cl. C12Q 1/00; C12Q 1/60

(52) U.S. Cl. 435/11; 435/4

(57) ABSTRACT

An object of the present invention is to provide a novel cholesterol detection reagent comprising a substance which can specifically binds to cholesterol to detect it, and a method for detecting cholesterol using the reagent. The present invention provides a cholesterol detection reagent comprising a polyethylene glycol cholesteryl ether which may be labeled.

### TECHNICAL FIELD

The present invention relates to a cholesterol detection reagent, and a method for detecting cholesterol using the reagent. More specifically, the present invention relates to a cholesterol detection reagent which comprises a polyethylene glycol cholesteryl ether, and a method for detecting cholesterol using the reagent.

### BACKGROUND ART

The content and distribution of intracellular cholesterol is stringently regulated. Inside the cells, cholesterol is accumulated in the post Golgi membranes (M. S. Bretscher, et al., Science 261,1280-1.(1993)). On the plasma membrane, cholesterol forms microdomains together with sphingomyelin and glycosphingolipids (A. Rietveld, et al., Biochim Biophys Acta 1376,467-79.(1998) ; and R. E. Brown, J Cell Sci 111,1-9.(1998)). Caveolins and other classes of proteins such as glycosylphosphatidylinositol (GPI)-linked glycoproteins and dually acylated non-receptor tyrosine kinases are located in these domains (T. V. Kurzchalia, et al., Curr Opin Cell Biol 11,424-31.(1999) ; and E. Ikonen, et al., Traffic 1,212-7.(2000)). These domains are known as lipid rafts. Lipid rafts are postulated to play an important role in cellular functions such as signaling, adhesion, motility, and membrane traffic (D. A. Brown, et al., Annu Rev Cell Dev Biol 14,111-36(1998); and K. Simons, et al., Nat Rev Mol Cell Biol 1,31-9.(2000)). Reduction of cellular cholesterol contents by removing surface cholesterol with methl-β-cyclodextrin (M β CD) or by metabolic inhibitors results in disintegration of these domains (L. J. Pike, et al., J. Biol Chem 273,22298-304.(1998) ; A. Pralle, et al., J Cell Biol 148,997-1008.(2000) ; and K. Roper, et al., Nat Cell Biol 2,582-92.(2000)).

Cellular content of cholesterol is controlled via the balance of de novo synthesis and exogenously obtained cholesterol through the endocytosis of lipoproteins (M. S. Brown, et al., Proc Natl Acad Sci USA 96,11041-8.(1999) : K. Simons, et al., Science 290,1721-6.(2000) ; and Y. A. Ioannou, Nat Rev Mol Cell Biol 2,657-68.(2001)). The collapse of this control leads to pathogenic conditions such as arteriosclerosis or Niemann-Pick type C (NPC) (P. G. Pentchev et al., Biochim Biophys Acta 1225,235-43.(1994) ; and L. Liscum, Traffic 1,218-25.(2000)). Internal membrane domains of late endosomes rich in lysobisphosphatidic acid are implicated in regulation of cholesterol transport by acting as a collection and distribution device (T. Kobayashi et al., Nat Cell Biol 1,113-8.(1999)). However, little is known about the intracellular transport of cholesterol and/or cholesterol-rich membrane domains.

Poly(ethylene glycol)cholesteryl ethers (PEG-Chols) are an unique group of nonionic amphiphatic molecules consisting of hydrophobic cholesteryl and hydrophilic poly(ethylene glycol) moieties (FIG. 1A) (H. Ishiwata, et al., Biochim Biophys Acta 1359,123-35(1997)). When added to living cells in culture, PEG(50)-Chol (moleculaw weight is 2587; 50 (in parentheses) is the number of ethylene glycol repeat) inhibited clathrin-independent, caveolac-like endocytosis under the condition of which clathrin-mediated internalization of transferrin was not affected (T. Baba et al., Traffic 2,501-12.(2001)). However, it remains unknown what type of cell components the PEG-Chol interacts with.

### DISCLOSURE OF THE INVENTION

It is an object of the present invention to identify a molecule to which a polyethylene glycol cholesteryl ether can specifically bind in cells. Further, it is another object of the present invention to provide a novel cholesterol detection reagent comprising a substance which can specifically binds to cholesterol to detect it, and a method for detecting cholesterol using the reagent.

The present inventors have carried out intensive studies to achieve the aforementioned objects. Taking into consideration the previous findings that PEG(50)-Chol specifically inhibits clathrin-independent endocytosis, the present inventors have assumed that PEG-Chol can specifically interact with one or more Lipid raft components, and have confirmed by overlay assay that PEG-Chol binds to various lipids in vitro. Moreover, as a result of studies regarding a substance with which PEG-Chol interacts in cells, the present inventors have found that PEG-Chol can specifically bind to cholesterol. The present invention has been completed based on these findings.

Thus, the present invention provides a cholesterol detection reagent comprising a polyethylene glycol cholesteryl ether which may be labeled.

In another aspect of the present invention, there is provided a method for detecting cholesterol, wherein a polyethylene glycol cholesteryl ether which may be labeled is used.

In the present invention, it is preferable to use a polyethylene glycol cholesteryl ether, which is labeled with an affinity substance or fluorescent substance.

### BRIEF DESCRIPTION OF THE DRAWINGS

FIG. 1 shows the results of an in vitro binding experiment using PEG-Chol.

FIG. 2 shows the results of a labeling experiment with PEG-Chol using cells. The bar indicates 20 μm.

FIG. 3 shows the results obtained by examining the distribution of fPEG-Chol on the surface of cells.

FIG. 4 shows the results obtained by examining the distribution of fPEG-Chol on the surface of cells.

FIG. 5 shows the results obtained by examining the distribution of fPEG-Chol on the surface of cells.

FIG. 6 shows the results obtained by analyzing the intra-membrane distribution of cholesterol and the fate of cholesterol on the surface of cells.

FIG. 7 shows the results obtained by analyzing the intra-membrane distribution of cholesterol and the fate of cholesterol on the surface of cells.

FIG. 8 shows the results obtained by analyzing the intra-membrane distribution of cholesterol and the fate of cholesterol on the surface of cells.

### BEST MODE FOR CARRYING OUT THE INVENTION

The embodiments of the present invention will be described below.

The cholesterol detection reagent of the present invention comprises a polyethylene glycol cholesteryl ether, which may be labeled.

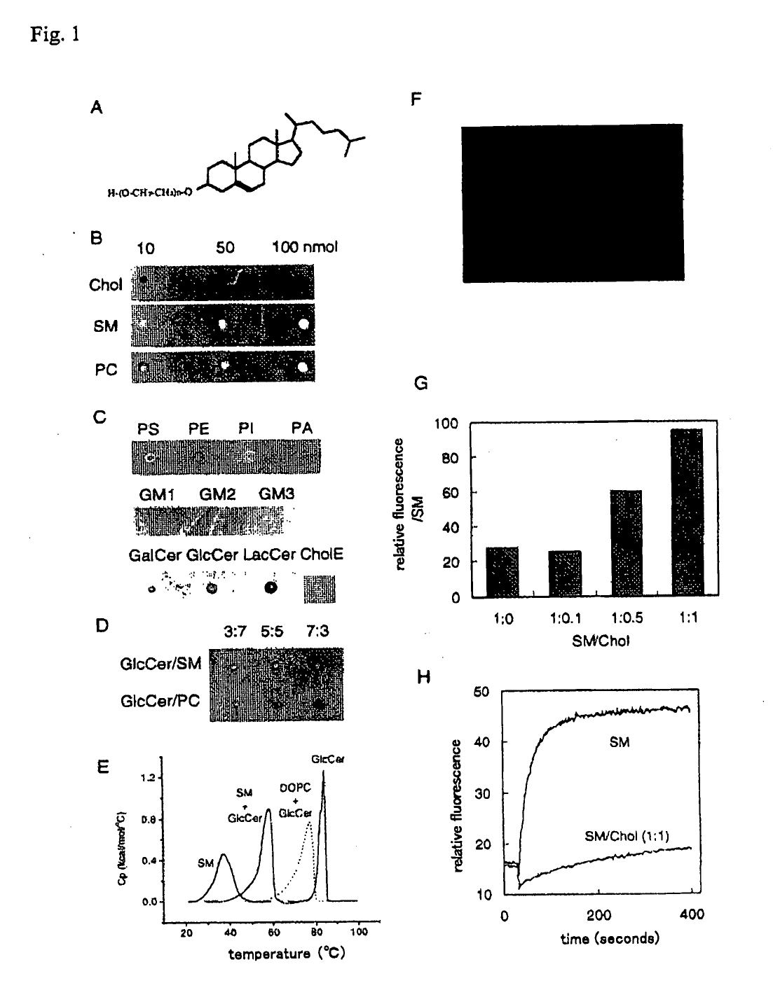

The polyethylene glycol cholesteryl ether used in the present invention is a compound having the structure shown in FIG. 1A, which consists of a hydrophobic cholesteryl moiety and a hydrophilic polyethylene glycol moiety (H. Ishiwata, et al., Biochim Biophys Acta 1359, 123-35 (1997)). In the structure, n represents the repeated number of ethylene glycols in the polyethylene glycol moiety. The number of n in the polyethylene glycol cholesteryl ether used in the present invention is not particularly limited, as long as it does not affect adversely the binding ability with cholesterol. For example, the number of n is between 10 and 1,000, preferably between 20 and 200, and more preferably between 20 and 100. An example of a preferably used compound may include a polyethylene glycol cholesteryl ether containing a polyethylene glycol moiety where n=50.

The polyethylene glycol cholesteryl ether used in the present invention is a known compound, which is, for example, described in the aforementioned publication (H. Ishiwata et al., Biochim Biophys Acta 1359, 123-35 (1997)). The polyethylene glycol cholesteryl ether used in the present invention can be produced by dissolving cholesterol in a solvent and injecting ethylene glycol gas into the obtained solution so as to perform a reaction (Ishiwata et al., Chem Pharm Bull 43, 1005-1011 (1995)). Other than this method, the polyethylene glycol cholesteryl ether can also be produced by a method involving allowing toluenesulfonate of cholesterol to react with polyethylene glycol (Patel et al., Biochim Biophys Acta 797: 20-26 (1984)).

As a polyethylene glycol cholesteryl ether used in the present invention, those to which a labeling substance used for detection binds are preferably used. The type of such a labeling substance is not particularly limited. Examples of such a labeling substance may include an affinity substance, a fluorescent substance, and a radioactive substance.

Examples of an affinity substance used herein may include biotin and digoxigenin. Examples of a fluorescent substance used herein may include fluorescein, FITC, BODIPY 493/503, BODIPY FL, dialkylaminocoumarin, 2′,7′-dichlorofluorescein, hydroxycoumarin, methoxycoumarin, naphthofluorescein, Oregon Green 514, tetramethylrhodamine (TMR), X-rhodamine, NBD, TRITC, Texas, Cy5, Cy7, IR144, FAM, JOE, TAMRA, and ROX. Examples of a radioactive substance used herein may include ³²P, ¹³¹I, ³⁵S, ⁴⁵Ca, ³H, and ¹⁴C. Other than these substances, oxidation stress-detecting agents such as carboxy-PTIO and DTCS (Dojin), NO-generating agents such as BNN5 (Dojin), various caged amino acids, chelating agents (e.g. DTPA, EDTA, NTA, etc.), and various carboxy disulfides (having the structure of (carboxylic acid) S—S (carboxylic acid)) may also be used.

The form of the cholesterol detection reagent of the present invention is not particularly limited, as long as it contains the aforementioned polyethylene glycol cholesteryl ether which may be labeled. The form may be either a solid or a liquid (a solution, a suspension, etc.). When cholesterol detection reagent is in the form of a liquid, the polyethylene glycol cholesteryl ether is dissolved or suspended in a suitable solvent (which is preferably an organic solvent or the like, regarding which the polyethylene glycol cholesteryl ether exhibits a certain degree of solubility), so as to prepare the reagent. To the reagent of the present invention, which is provided in the aforementioned form, assistant agents other than the polyethylene glycol cholesteryl ether (e.g. a preservative, a stabilizer, a pH buffer, etc.) can also be added as appropriate.

The present invention also provides a method for detecting cholesterol using the polyethylene glycol cholesteryl ether which may be labeled. Detection may be carried out in vitro, in a cell, or in vivo. First, a specimen containing cholesterol to be detected is allowed to come into contact with a polyethylene glycol cholesteryl ether (which is preferably labeled) under certain conditions, so as to bind them to each other.

After completion of the binding, the polyethylene glycol cholesteryl ether which was bound to cholesterol is detected. Detection can appropriately be carried out depending on the type of the label used.

When biotin is used as a label for example, detection can be carried out using avidin or streptavidin, which specifically bind to biotin. For example, a biotin-labeled polyethylene glycol cholesteryl ether which was bound to cholesterol is allowed to react with avidin or streptavidin, and a biotinated alkaline phosphatase is then allowed to bind thereto, so that the enzyme binds thereto via biotin. After an unbounded enzyme portion has been removed, nitroblue tetrazolium (NBT), which is a substrate of alkaline phosphatase, is allowed to react with 5-bromo-4-chloro-3-indolylphosphate (BCIP). As a result, when a biotin-labeled polyethylene glycol cholesteryl ether exists, the development of a violet color is seen, and it can therefore be detected. When digoxigenin is used as a label, detection can be carried out using an alkaline phosphatase-labeled anti-digoxigenin antibody by the same method as described above. Other than alkaline pbosphatase, a system using horseradish peroxidase has also been known as an enzyme used for color development.

When a fluorescent substance such as a fluorescein is used, a polyethylene glycol cholesteryl ether which was bound to cholesterol can be detected by measuring fluorescence after completion of the reaction with cholesterol. That is, fluorescence energy generated as a result of application of a certain amount of excitation light is measured, so as to qualitatively or quantitatively detect fluorescence. When fluorescence is quantitatively detected, the intensity of fluorescence energy can be evaluated as an indicator of the abundance of cholesterol. Such fluorescence energy or fluorescence can be measured using a suitable detector or fluorescence microscope, which are commercially available.

When a radioactive substance is used, after completion of the reaction with cholesterol, radioactivity which was bound to the cholesterol is measured by a method known to a person skilled in the art, so as to detect the cholesterol.

The present invention will be more specifically described in the following examples. However, the examples are not intended to limit the scope of the present invention.

### EXAMPLES

### EXAMPLE 1

### In Vitro Binding Experiment using PEG-Chol

(Methods)

(1) The binding ability of biotinylated PEG-Chol (bPEG-Chol: one molecule of biotin is conjugated to the terminal ethylene glycol moiety of PEG(50)-Chol) (10 μM) to various amounts of lipids was analyzed by overlay assay, which was performed on TLC plates, as described in the previous report (K. Igarashi et al., J Biol Chem 270, 29075-8. (1995)). The results are shown in FIG. 1B.
(2) The binding of bPEG-Chol (10 μM) to various lipids, glycolipids, and cholesterol oleate (100 nmol) was examined in the same manner as described in (1) above. The results are shown in FIG. 1C.
   (3) The binding of bPEG-Chol to a mixture consisting of glucosylceramide (GlcCer) and sphingomyelin (SM) or a mixture consisting of glucosylceramide and dioleoylphosphatidylcholine (DOPC) (total 30 nmol with the ratio indicated in FIG. 1D) was analyzed. The results are shown in FIG. 1D.
(4) The traces of thermograms obtained by differential scanning calorimetry performed on GlcGer, SM, GlcCer+SM (1:1), and GlcCer+DOPC (1:1) were measured. 500 μl of a suspension containing 1 mM liposomes (GlcCer, SM, and DOPC) or 2 mM liposomes (GlcCer+SM and GlcCer+DOPC) was measured using MicroCal VP-DSC. The results are shown in FIG. 1E.
(5) The fluorescence image of a monolayer composed of a mixture consisting of GlcCer and DOPC at a ratio of 1:1 was obtained. A lipid monolayer was prepared by injecting 20 μl of a chloroform solution of 1 mM GlcCer+DOPC containing 0.5% C12-BODIPY-PC (Molecular Probes) into a USI system (Fukuoka, Japan) FSD-500 Langmuir-Blodgett trough. The C12-BODIPY-PC was preferentially partitioned into the DOPC phase. The surface pressure was adjusted to 10 mM/m. Using an Olympus Power BX fluorescent microscope equipped with an LM Plan FI 50× objective and a Toshiba 3CCD camera, a fluorescence image was recorded. The results are shown in FIG. 1F. The bar indicates 50 μm.
(6) Using 1 mM sphingomyelin vesicles containing various amounts of cholesterols, the binding of fluorescein PEG-Chol (fPEG-Chol) containing a fluorescein on the distal end of a PEG chain was analyzed (H. Ishiwata et al., Biochim Biophys Acta 1359, 123-35 (1997)). Vesicles were produced in the manner described in the previous report (A. Miyazawa et al, Mol Immunol 25, 025-31. (1988)). Vesicles were incubated with fPEG-Chol at room temperature for 30 minutes. Unbounded fPEG-Chol was washed by centrifugation at 15 K×g for 15 minutes. The fluorescence of the pellet was measured, and normalized with phosphorus of sphingomyelin. The results are shown in FIG. 1G.
(7) Transfer of fPEG-Chol between membranes was analyzed. 500 μM (final concentration) SM/Chol (1:1) liposomes were added to liposomes (50 μM) composed of SM alone or SM/Chol (1:1), which contained 0.5 μM fPEG-Chol and 0.5 μM N-rhodamine-dipalmitoylphosphatidylethanolamine. The release of fluorescence resonance energy transfer (FRET) was measured by monitoring time course of fluorescence emission spectrum at 535 nm with excitation at 488 nm. The results are shown in FIG. 1H.

It is to be noted that cholesterol and cholesterol oleate were purchased from Sigma (St. Louis, Mo.). Galactosylceramide, glucosylceramide, and lactosylceramide were purchased from Matreya (State College, Pennsylvania). All other lipids were purchased from Avanti Polar lipids (Alabaster, Ala.).

Chol represents cholesterol, SM represents sphingomyelin, PC represents phosphatidylcholine, PS represents phosphatidylserine, PE represents phosphatidylethanolamine, PI represents phosphatidylinositol, PA represents phosphatidic acid, GM1 represents ganglioside GM1, GM2 represents ganglioside GM2, GM3 represents ganglioside GM3, GalCer represents galactosylceramide, GlcCer represents glucosylceramide, and LacCer represents lactosylceramide.

(Results)

Biotinylated PEG-Chol (bPEG-Chol: one molecule of biotin is conjugated to the terminal ethylene glycol moiety of PEG(50)-Chol) was added to spots of various lipids. After washing, the binding was monitored by HRP-conjugated streptavidin using 4-chloro-1-naphtol as a substrate (FIGS. 1B and 1C) (A. Yamaji et al., J Biol Chem 273, 5300-6. (1998)). The bPEG-Chol bound to cholesterol and neutral glycolipids (e.g. galactosylceramide, glucosylceramide (GlcCer), and lactosylceramide). However, the bPEG-Chol did not bind to phospholipids and acidic glycolipids (gangliosides) tested. Also, it did not bind to cholesteryl ester and cholesterol oleate. Moreover, the addition of sphingomyelin (SM) abolished the binding of bPEG-Chol to glucosylceramide, but sphingomyelin (SM) did not have such effects on dioleoylphosphatidylcholine (DOPC) (FIG. 1D).

Differential scanning calorimetry (DSC) showed that an equimolar mixture consisting of SM and GlcCer gave a gel-to-liquid crystalline phase transition temperature in the middle of those of SM and GlcCer (FIG. 1E). In contrast, the phase transition temperature of an equimolar mixture consisting of DOPC and GlcCer was very close to that of GlcCer, whereas the phase transition temperature of DOPC was much lower than that of SM. These results suggest that GlcCer is miscible with SM whereas a binary mixture consisting of this lipid and DOPC is segregated in different domains.

In order to confirm that GlcCer is segregated from DOPC, a monolayer system was employed (FIG. 1F). A monolayer experiment clearly showed that GlcCer (black) was segregated from DOPC (green) to form domains at an air-water interphase. These results suggest that PEG-Chol binds to neutral glycolipids only when they are clustered each other. The detergent solubility of cell membranes (D. A. Brown et al., Cell 68, 533-44. (1992)) and the measurement of lipid partitioning in model membranes (T. Y. Wang et al., Biophys J 79, 1478-89. (2000)) suggest that glycolipids are distributed to sphingomyelin-rich membranes in cells. Taking into account the high concentration of sphingomyelin in biomembranes, these results suggest that PEG-Chol may not significantly bind to glycolipids in cells. In contrast to glycolipids, the addition of sphingomyelin did not affect bPEG-Chol binding to cholesterol until the cholesterol content was reduced to less than 10%.

In order to examine the binding of PEG-Chol to cholesterol, a liposome experiment was further conducted using fluorescein PEG-Chol (fPEG-Chol) containing a fluorescein on the distal end of a PEG chain. As in the case of overlay assay, the addition of cholesterol increased the binding of fPEG-Chol to sphingomyelin liposomes (FIG. 1G). The fact that fPEG-Chol did not bind to SM liposomes when the cholesterol content was low (10%) suggests that fPEG-Chol recognizes cholesterol-rich domains in the aforementioned membranes.

PEG-Chol is water-soluble and can be transferred between membranes. In FIG. 1H, the transfer of fPEG-Chol between membranes was measured. In order to measure the transport of fPEG-Chol, fluorescence resonance energy transfer (FRET) between fPEG-Chol and rhodamine-labeled phosphatidylethanolamine (rhodamine-PE) used as a non-exchangeable marker was measured (J. W. Nichols et al., Biochemistry 21, 1720-6. (1982)). In donor liposomes, fPEG-Chol fluorescence was quenched by FRET. However, once fPEG-Chol was transported to acceptor liposomes, fluorescence was de-quenched. When SM liposome was used as a donor and SM/Chol (1:1) liposome was used as an acceptor, the efficient transport of fPEG-Chol was observed. In contrast, when both donor and acceptor were SM/Chol (1:1), fPEG-Chol did not transfer significantly. These results indicate that PEG-Chol is preferentially incorporated into cholesterol-rich membranes, and that once it is incorporated therein, it is trapped in the membranes.

### Example 2

### Labeling Experiment using Cells Labeled with PEG-Chol

(Methods)

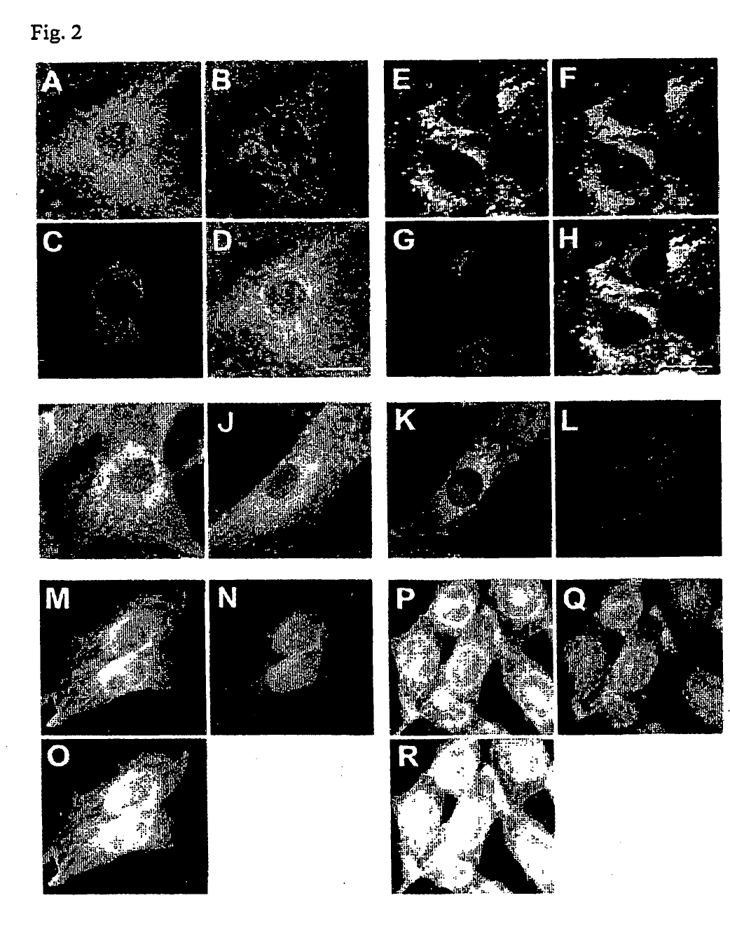

As described in the previous report (T. Kobayashi et al., Nat Cell Biol 1, 113-8. (1999)), normal (FIGS. 2A to 2D) and NPC (FIGS. 2E to 2H) human skin fibroblasts were fixed and permeabilized. Cells were then triply labeled with 5 μM fPEG-Chol (FIGS. 2A and 2E), 50 μg/ml filipin (FIG. 2B and 2F), and an anti-TGN 46 antibody (Serotec Inc., Oxford, U.K.) (FIGS. 2C and 2G). The specimens were observed using a Zeiss LSM confocal microscope. FIGS. 2D and 2H show merged images. White color indicates the co-localization of 3 types of fluorophores. With regard to the specimens stained with fPEG-Chol and filipin, normal cells and NPC cells were exposed to the laser light differently since the fluorescence is much brighter in NPC cells.

In FIG. 2I and 2J, NPC cells were allowed to grow in the presence of normal serum (FIG. 2I) or delipidated serum (FIG. 2J). Thereafter, the cells were permeabilized and labeled with fPEG-Chol.

In FIG. 2K and 2L, NPC skin fibroblasts were fixed and permeabilized. Thereafter, the cells were labeled with fPEG-Chol in the presence of 1 mM sphingomyelin liposomes (FIG. 2K) or sphingomyelin/cholesterol (1:1) liposomes (FIG. 2L).

In FIGS. 2M to 2R, a melanoma cell line MEB4 (FIGS. 2M to 2O) and a mutant GM95 that is a melanoma cell line defective in glycolipid synthesis (FIGS. 2P to 2R) were fixed and permeabilized. Thereafter, the cells were doubly labeled with fPEG-Chol (FIGS. 2M and 2P) and filipin (FIGS. 2N and 2Q). Similar fluorescence pattern in MEB4 and GM95 suggests that the labeling with fPEG-Chol is not primarily dependent on glycolipids. fPEG-Chol labeling was co-localized with filipin labeling (FIGS. 2O and 2R).

(Results)

The in vitro interaction of PEG-Chol and various lipids suggests that this molecule will be incorporated into specific cholesterol-rich membranes or membrane domains in the cell, When fPEG-Chol was added to permeabilized human skin fibroblasts, the Golgi apparatus emitted bright fluorescence (FIG. 2A). A similar but less clear pattern of fluorescence had previously been observed when filipin forming a complex with cholesterol had been used (J. Sokol et al., J Biol Chem 263, 3411-7. (1988); and T. Kobayashi et al., Nat Cell Biol 1, 113-8. (1999)). fPEG-Chol staining was partially co-localized with a trans-Golgi network marker, TGN46 (A. R. Prescott et al., Eur J Cell Biol 72, 238-46. (1997)). Incomplete overlap suggests that TGN46 and cholesterol are differently distributed in the Golgi apparatus. Niemann-Pick type C (NPC) is an autosomal recessive, neurovisceral disease. The hallmark of the NPC syndrome is the intracellular accumulation of unesterified cholesterol (P. G. Pentchev et al., Biochim Biophys Acta 1225, 235-43. (1994); L. Liscum, Traffic 1, 218-25. (2000); and T. Kobayashi et al., Nat Cell Biol 1, 113-8. (1999)). Differing from normal fibroblasts, fPEG-Chol stains perinuclear vesicles as well as the Golgi apparatus in NPC fibroblasts (FIG. 2E). In this case also, the fluorescence was co-localized with filipin (FIGS. 2F and 2H).

Cholesterol accumulation was significantly decreased when NPC cells were allowed to grow in the absence of lipoproteins (J. Sokol et al., J Biol Chem 263, 3411-7. (1988)). When NPC cells were allowed to grow in the presence of delipidated serum instead of normal serum, perinuclear labeling with fPEG-Chol was dramatically decreased (FIGS. 2I and 2J). When fPEG-Chol was preincubated with SM/Chol (1:1) liposomes, fPEG-Chol labeling was abolished (FIG. 2L). Cholesterol-free sphingomyelin liposomes showed much fewer effects under the same conditions (FIG. 2K). Once incorporated in membrane domains, fPEG-Chol was not removed therefrom even using SM/Chol liposomes. This strengthens the idea that fPEG-Chol is trapped in cholesterol-rich membrane domains in the cell.

GM95 is a melanoma cell line defective in glycolipid synthesis (S. Ichikawa et al., Proc Natl Acad Sci USA 91, 2703-7. (1994)). In order to examine the effects of glycolipids on PEG-Chol staining, GM95 was compared with parent MEB4 cells. Both GM95 and MEB4 were labeled with fPEG-Chol in similar manners (FIGS. 2M and 2P). In addition, this labeling was co-localized with filipin labeling. These results suggest that the labeling of cells with fPEG-Chol was primarily dependent on cellular cholesterol but not on glycolipids.

### Example 3

### Distribution of fPEG-Chol on Cell Surface

(Methods)

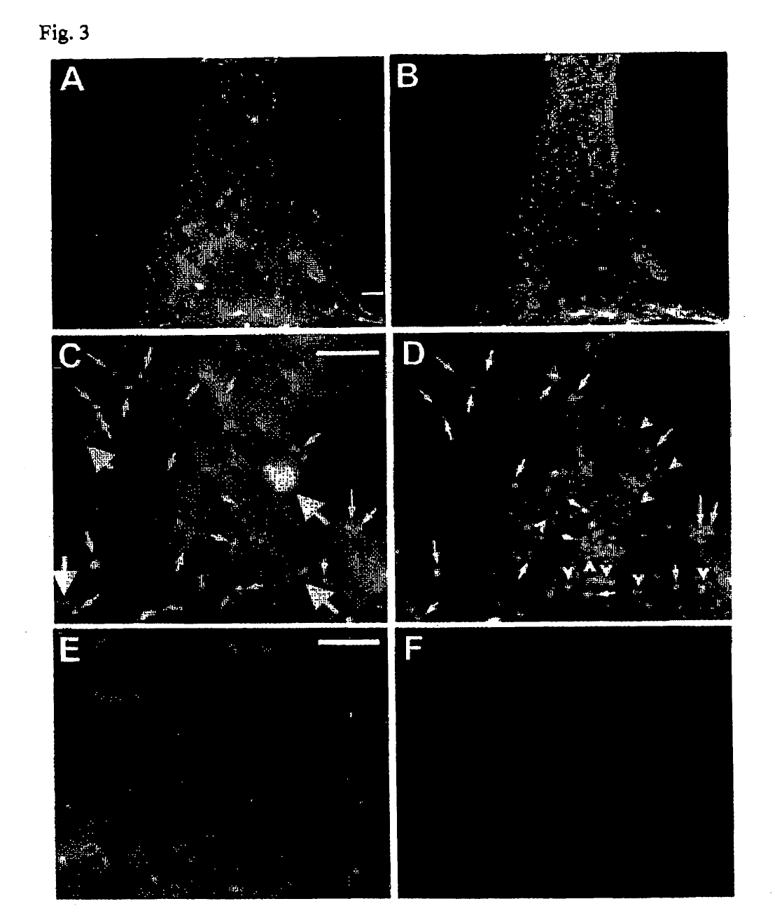

Normal human skin fibroblasts were incubated together with cholera toxin labeled with 1 μM fPEG-Chol and 5 μM AlexaFluor 594 at room temperature for 90 seconds. Thereafter, the cells were fixed with paraformaldehyde for 10 minutes. FIGS. 3A and 3C show fPEG-Chol fluorescence, and FIGS. 3B and 3D show AlexaFluor 594 fluorescence. Small arrows indicate structure, which were double-labeled with fPEG-Chol and cholera toxin. Large arrows indicate those labeled only with fPEG-Chol. Arrowheads indicate the spots that are positive with cholera toxin alone. In FIGS. 3E and 3F, before fixation, the cells were treated with (E) and without (P) 10 mM MβCD at 37° C. for 30 minutes. Thereafter, the cells were labeled with 1 μM fPEG-Chol. In FIG. 3, the bar indicates 4 μm.

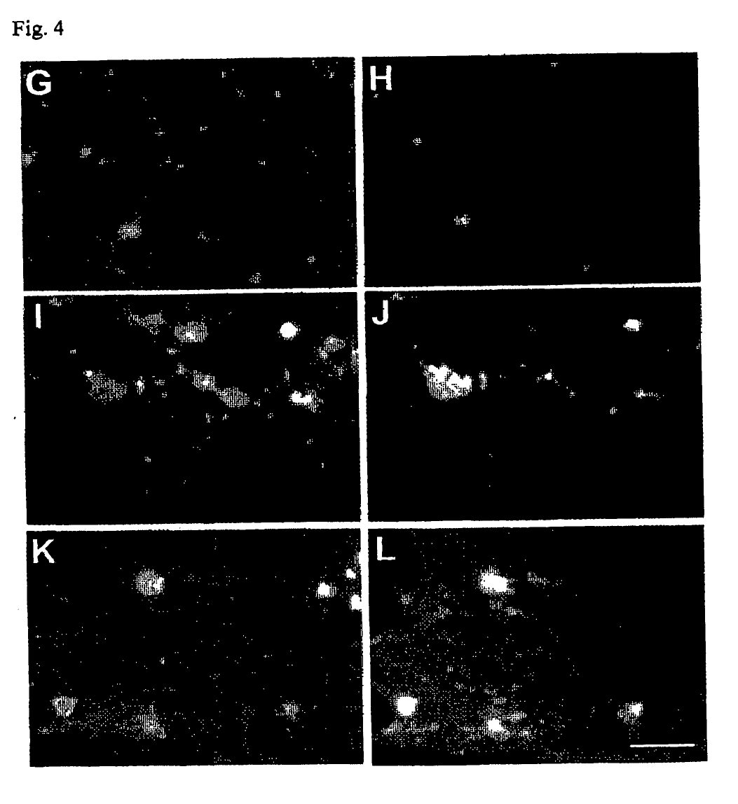

In FIGS. 4G to 4L, normal skin fibroblasts were labeled with 2 μM fPEG-Chol. Thereafter, the cells were incubated with a 5 μg/ml biotinylated epidermal growth factor (EGF) at 4° C. for 20 minutes (FIGS. 4G and 4H), or at 37° C. for 2 minutes (FIGS. 4I and 4L). Thereafter, the cells were fixed with PBS containing 3% PFA and 8% sucrose, quenched, and then incubated with TRITC-labeled streptavidin at 4° C. for 20 minutes. The specimens were observed with a Nikon TE 300 microscope equipped with a Hamamatsu C-4742-98 cooled CCD camera. In FIG. 4, G and I indicate fPEG-Chol fluorescence, and H and J indicate AlexaFluor 594 EGF-fluorescence. In K and L in FIG. 4, the cells were doubly labeled with 1 μM fPEG-Chol and an AlexaFluor 594-labeled cholera toxin B subunit prior to being stimulated by non-labeled EGF. In FIG. 4, K indicates fPEG-Chol fluorescence, and L indicates cholera toxin fluorescence. In FIG. 4, the bar indicates 4 μm.

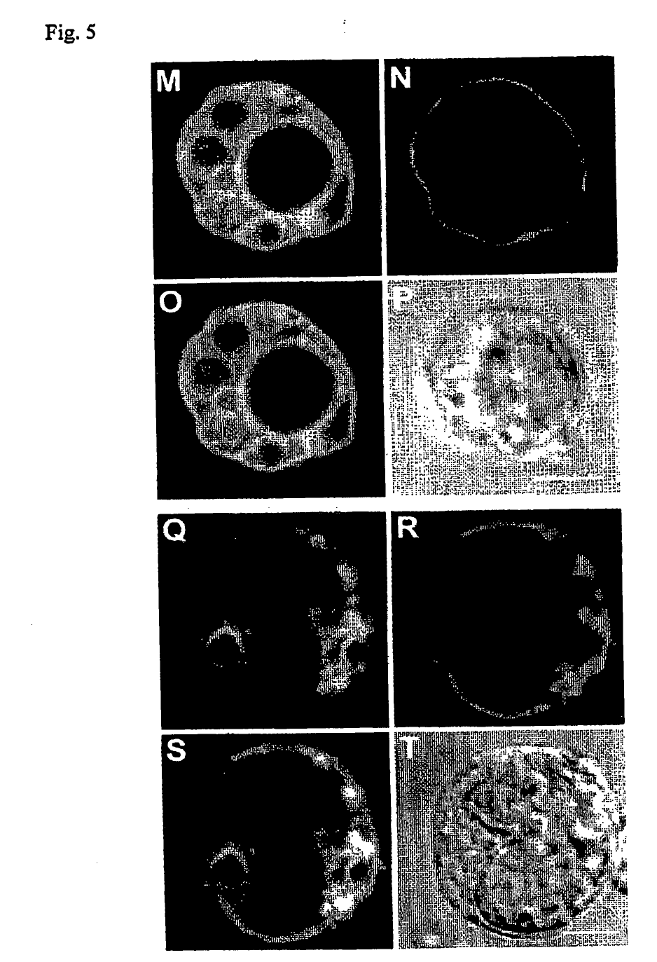

In M to P in FIG. 5, B cell line A20.2J was incubated at 37° C. for 1 minute without antibodies. Cells were then washed and fixed with 1% PFA for 30 minutes, and then labeled with 0.7 μM fPEG-Chol and a 10 μg/ml Alexa 546-conjugated cholera toxin B subunit in 0.1% BSA on ice for 45 minutes. After washing, the stained cells were observed under a Zeiss LSM 510 confocal microscope. In FIG. 5, M indicates fPEG-Chol labeling, N indicates cholera toxin labeling, O indicates a merged image, and P indicates a phase contrast image. Under these conditions, fPEG-Chol permeates the fixed cells, so as to stain intracellular membranes as well as plasma membranes. In contrast, cholera toxin did not enter the cells, and thus, it stained only the cell surfaces.

In Q to T in FIG. 5, A20.2J cells were stimulated with 15 μg/ml F(ab′)₂ goat antibodies specific for mouse IgG+IgM (F(ab′)₂ anti-Ig) at 37° C. for 1 minute. Thereafter, the cells were fixed and stained as described above. In FIG. 5, Q indicates fPEG-Chol labeling, R indicates cholera toxin labeling, S indicates a merged image, and T indicates a phase contrast image.

(Results)

In Example 3, the distribution of fPEG-Chol on the cell surface was examined (FIGS. 3 to 5).

Normal human skin fibroblasts were treated with fPEG-Chol, and then washed and fixed. Non-uniform surface labeling with higher fluorescence were observed in small domains (with diameters between 200 and 500 nm) (FIG. 3A and 3C). Some of these domains were co-localized with an AlexaFluor 594-labeled cholera toxin B chain (FIGS. 3B and 3D). Cholera toxin binds to GM1, which is non-randomly distributed on the plasma membranes and accumulates in caveolae (R G. Parton, J Histochem Cytochem 42, 155-66. (1994)). When the cells were pretreated with methyl-p-cyclodextrin (MβCD), which specifically removes cholesterol from cells, fPEG-Chol staining disappeared (FIG. 3E and 3F) (G. H. Rothblat et al., J Lipid Res 40, 781-96. (1999)).

Subsequently, the distribution of fPEG-Chol when cells were not stimulated with an epidermal growth factor (EGF) was measured. An EGF receptor localized to cholesterol-rich plasma membrane domains, and thus, it was suggested that the binding of EGF to the EGF receptor is dependent on cell surface cholesterol (M. G. Waugh et al., Biochem Soc Trans 29, 509-11. (2001): K. Roepstorff et al., J Biol Chem 8, 8 (2002); and T. Ringerike et al., J Cell Sci 115, 1331-40. (2002)). fPEG-Chol fluorescence was co-localized with the distribution of biotin-labeled EGF, when EGF was added at 4° C. (FIGS. 4G and 4H). When EGF was added at 37° C., the clustering of EGF receptors was observed (FIG. 4J). These clusters were labeled with fPEG-Chol (FIG. 4I). The cell surface distribution of GM1 was also examined under these conditions. GM1 was also enriched in these clusters and further co-localized with fPEG-Chol (FIGS. 4K and 4L). These results indicate that EGF induces re-distribution of both cholesterol and GM1 to the same clusters where EGF receptors were enriched.

Re-distribution of plasma membrane ganglioside occurs during the cross-linking of B cell antigen receptors on the plasma membrane of a B cell line A20.2J (M. J. Aman et al., J Biol Chem 276, 46371-8. (2001)). Whether or not fPEG-Chol is re-distributed by treatment with F(ab′)₂ anti Ig was examined. Before the treatment, both AlexaFluor 594-labeled cholera toxin and fPEG-Chol outlined the entire surface (FIGS. 5M to 5P). However, after stimulation with a F(ab′)₂ fragment for 1 minute, cholera toxin was accumulated in aggregated structures on the plasma membranes (FIG. 5R). fPEG-Chol also localized to these structures (FIGS. 5Q and 5S). These results indicate that cholesterol is re-distributed together with GM1 during stimulation of B cell lines.

### Example 4

### Analysis on Intra-Membrane Distribution of Cholesterol and Fate of Cell Surface Cholesterol

(Methods)

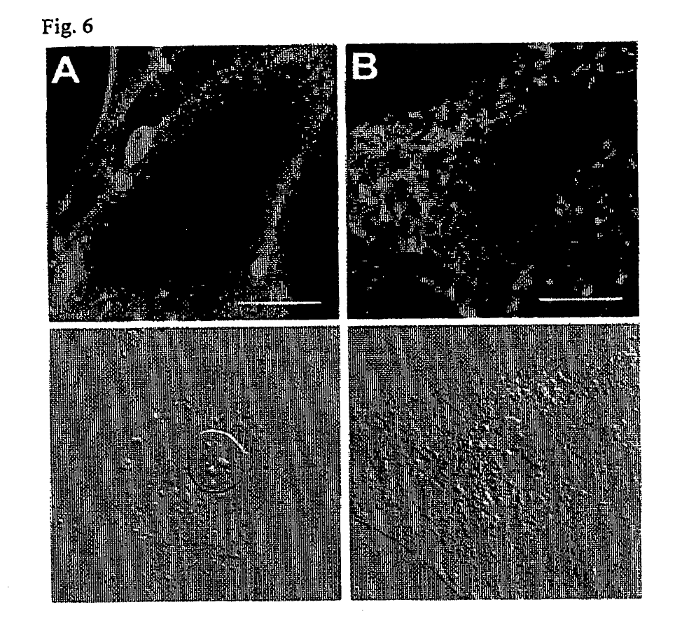

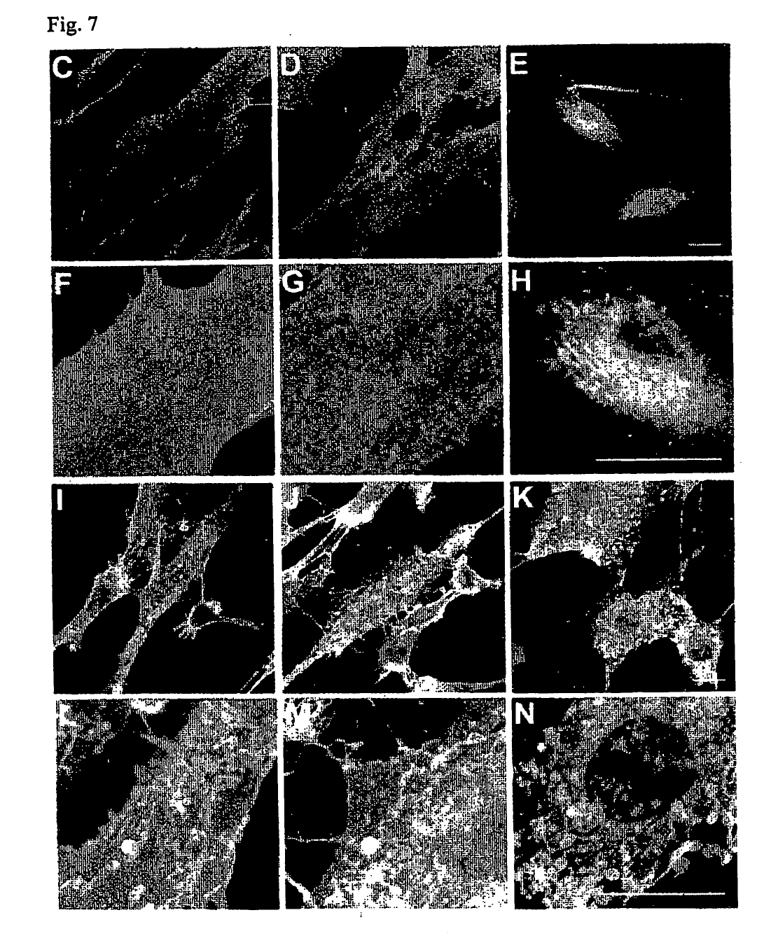

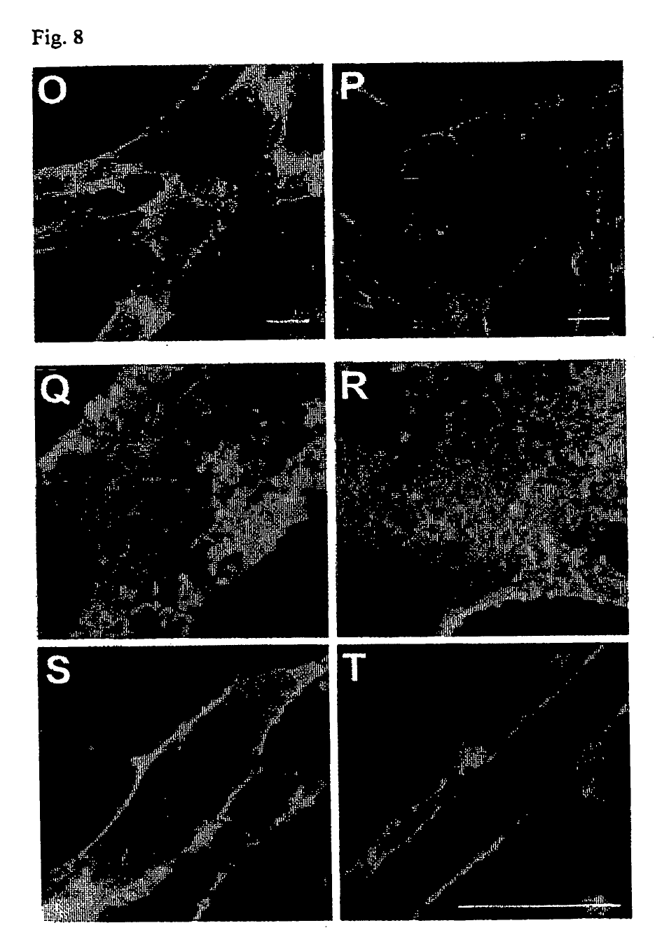

(1) As described above, the plasma membranes of normal (FIG, 6A) and NPC (FIG. 6B) fibroblasts were permeabilized using streptolysin O. The cells were incubated with fPEG-Chol at room temperature for 30 minutes before washing and taking fluorescence images under a Zeiss LSM 510 confocal microscope. The results are shown in FIG. 6.
(2) Normal (FIGS. 7C to 7H) and NPC (FIGS. 7I to 7N) fibroblasts were incubated with 1 μM fPEG-Chol at room temperature for 5 minutes. Cells were washed and incubated for 10 minutes (FIG. 7, F, L and L), 60 minutes (FIG. 7, D, G, J, and M), and 180 minutes (FIG. 7, E, H, K, and N) at 37° C. in the presence of 1 mg/ml rhodamine dextran. The results are shown in FIG. 7.
(3) NPC fibroblasts were incubated with 1 μM fPEG-Chol at room temperature for 5 minutes. Cells were then washed and incubated at 37° C. for 30 minutes (FIG. 8O). NPC fibroblasts were incubated with 1 μM fPEG-Chol at 4° C. for 30 minutes. Cells were then washed and photographed. Cells were then washed and incubated at 37° C. for 30 minutes (FIG. 8P). NPC fibroblasts were treated with 5 μg/ml brefeldin A for 30 minutes (FIG. 8Q), 5 82 g/ml nocodazole for 90 minutes (FIG. 8R), or 5 μg/ml cytochalasin B for 30 minutes (FIG. 8S) before incubation with 1 μM fPEG-Chol and 1 mg/ml rhodamine dextran for 30 minutes. In FIG. 8T, NPC fibroblasts were incubated with 1 μM fPEG-Chol for 30 minutes before treatment with 5 μg/ml cytochalasin B for 30 minutes. In FIGS. 6 to 8, the bar indicates 20 μm.
   (Results)

Little has been known about the intra-membrane distribution of cholesterol. In the present example, whether or not cholesterol is located in the cytoplasmic side or luminal side of the intracellular membranes was examined by using semi-permeable cells. Plasma membranes of normal and NPC skin fibroblasts were selectively permeabilized by bacterial toxin streptolysin O. Cells were then incubated with fPEG-Chol (FIGS. 6A and 6B). The fPEG-Chol staining was dramatically different from those obtained in fixed and permeabilized cells (FIGS. 2And 2E). In addition, there was a big difference between normal and NPC cells. In normal skin fibroblasts, peripheral vesicle-like structures were strongly stained, whereas in NPC cells, meshwork-Like structures were visualized. These structures were not observed after cells were fixed and permeabilized, suggesting that these compartments were either fragile or detergent sensitive. Golgi apparatus and late endosomes/lysosomes were not significantly labeled under these conditions. These results suggest that cholesterol resides only in the lumen of these organelles. In contrast, peripheral vesicles in normal fibroblasts and meshwork structures in NPC cells contain cholesterol in the cytoplasmic membranes.

The detailed mechanism(s) of the intracellular accumulation of free cholesterol in NPC cells is not well understood. Recent studies suggest that the accumulation results from an imbalance in the brisk flow of cholesterol among membrane compartments (Y. Lange et al., J Biol Chem 275, 17468-75. (2000)). Both the endogenously synthesized cholesterol and that derived via LDL once reach the plasma membrane, they are then internalized in the cell. Cruz et al. suggested that NPC1 (that is a protein encoded by the gene whose mutation is responsible for the disease) is involved in a post-plasma membrane cholesterol-trafficking pathway (J. C. Cruz et al., Biol Chem 275, 4013-21. (2000)). In order to chase the fate of cell surface cholesterol, filipin is not suitable because of the toxicity. A fluorescent cholesterol analog, dehydroergosterol, was shown to be endocytosed and accumulated in recycling compartment in a CHO cell line (S. Mukherjee et al., Biophys J 75, 1915-25. (1998); and M. Hao et al., J Biol Chem 277, 609-17. (2002)). DHE differs from cholesterol in having three additional double bonds and an extra methyl group. Recently, it has been shown that perfringolysin O binds selectively to cholesterol-rich membrane domains (A. A. Waheed et al., Proc Natl Acad Sci USA 98, 4926-31. (2001); and W. Mobius et al., J Histochem Cytochem 50,43-55. (2002)). Advantages of using fPEG-Chol may include higher stability and quantum efficiency of the fluorophore, lower background staining, lower cell toxicity, and possibly minor structural perturbation at the working concentration because of the relatively small size.

The fate of cell surface fPEG-Chol of normal fibroblasts was compared with that of NPC fibroblasts (FIGS. 7C to 7N). In the present experiment, 1 μM fPEG-Chol was used. This concentration of fPEG-Chol did not affect the endocytosis of dextran and cholera toxin in this system. Cells were incubated with fPEG-Chol at room temperature for 5 minutes, washed, and further incubated at 37° C. in the presence of 1 mg/ml rhodamine dextran. In normal fibroblasts, cell surface was strongly labeled after 5 minutes of fPEG-Chol labeling. Most of the fluorescence stayed on the plasma membrane after 10 minutes of chase (FIGS. 7C and 7F). After 60 minutes of chase, nucleus became recognized as a non-labeled organelle surrounded by cytoplasmic fluorescent compartments (FIG. 7D). The overall pattern of these compartments was similar to that detected by DHE-MβCD in CHO cells (M. Hao et al., J Biol Chem 277, 609-17. (2002)). However, fPEG-Chol also stained intracellular vesicles. Most of these vesicles were not co-localized with internalized rhodamine dextran (FIG. 7G). These vesicles are often observed in the periphery of cells, like those observed in FIG. 6A. After 180 minutes, Golgi apparatus was prominently labeled with fPEG-Chol while rhodamine fluorescence was distributed in endosomes/lysosomes (FIGS. 7E and 7H). The fate of fPEG-Chol was dramatically different in NPC fibroblasts. After 10 minutes of chase, fPEG-Chol stained characteristic meshwork structures (FIGS. 7I and 7L), which was never observed in normal cells. Even after 180 minutes of chase, most of the fPEG-Chol was retained in this structure and Golgi fluorescence was hardly visible (FIGS. 7J and 7M). Sometimes, internalized rhodamine dextran was surrounded by the meshwork structures (FIG. 7M, arrows), suggesting that these structures have characteristics of endocytic compartments. These structures are very similar to those observed in FIG. 6B.

The incorporation of fPEG-Chol into the meshwork structure is temperature dependent. At 4° C., fPEG-Chol stayed on the plasma membrane and was not incorporated into the meshwork (FIG. 8P). FIG. 8P also indicates that fPEG-Chol does not undergo spontaneous transbilayer movement. The fluorophores, which undergo spontaneous flip-flop, stain intracellular membranes under these conditions (R. E. Pagano et al., J Cell Biol 91, 872-7. (1981); and R. E. Pagano et al., J Biol Chem 260, 1909-16. (1985)). Subsequently, the internalization of fPEG-Chol and rhodamine dextran was measured in the presence of inhibitors. Brefeldin A (an inhibitor of post-Golgi transport and nocodazole, which inhibits microtubule assembly) did not significantly affect the incorporation of fPEG-Chol into meshwork. In contrast, meshwork structure was disappeared by cytochalasin B (which inhibits actin polymerization). Cytochalasin B did not affect the internalization of rhodamine dextran. In FIG. 8T, cells were labeled with fPEG-Chol before treatment with cytochalasin B. In this case also, the meshwork structure was disappeared, suggesting that the meshwork structure is dependent on action network.

### INDUSTRIAL APPLICABILITY

From the aforementioned results of the examples, it was demonstrated that fPEG-Chol is a useful means for visualizing cholesterol-rich domains. That is to say, the present invention provides a novel cholesterol detection reagent having advantages such as higher stability and quantum efficiency of the fluorophore, lower background staining, lower cell toxicity, and possibly minor structural perturbation at the working concentration because of the relatively small size.

1. A cholesterol detection reagent comprising a polyethylene glycol cholesteryl ether which may be labeled.

2. The cholesterol detection reagent according to claim 1 wherein the polyethylene glycol cholesteryl ether is labeled with an affinity substance or fluorescent substance.

3. A method for detecting cholesterol, wherein a polyethylene glycol cholesteryl ether which may be labeled is used.

4. The method for detecting cholesterol according to claim 3 wherein a polyethylene glycol cholesteryl ether which is labeled with an affinity substance or fluorescent substance is used.
